(19) United States
(12) Patent Application Publication
(10) Pub. No.: US 2026/0232457 A1
(43) Pub. Date: Aug. 13, 2026

(54) INTERVERTEBRAL FUSION CAGE WITH BIOMIMETIC TRABECULAR BONE AND FULLY POROUS STRUCTURE MANUFACTURED BY ADDITIVE MANUFACTURING

(71) Applicant: Shenzhen Dazhou Medical Technology Co., Ltd., Shenzhen (CN)

(72) Inventors: Jingzhou Yang, Shenzhen (CN); Dachen Zhang, Shenzhen (CN); Haishen Chen, Shenzhen (CN); Jinghao Yang, Shenzhen (CN)

(21) Appl. No.: 19/240,455

(22) Filed: Jun. 17, 2025

(30) Foreign Application Priority Data
Feb. 8, 2025 (CN) 2025101440416

(51) Int. Cl. A61F 2/44; A61F 2/30; A61F 2/46; B33Y 80/00

(52) U.S. Cl. CPC A61F 2/447; A61F 2/30771; A61F 2/3094; A61F 2/446; A61F 2/4611; A61F 2002/30477; A61F 2002/3092; A61F 2002/3093; A61F 2002/3097; A61F 2002/30985; B33Y 80/00

(57) ABSTRACT

An intervertebral fusion cage with a biomimetic trabecular bone and fully porous structure manufactured by additive manufacturing is applied in spinal fusion surgery, made of medical metal and manufactured by selective laser melting or electron beam melting. The intervertebral fusion cage includes: a front end surface, a rear end surface, a first side surface, a second side surface, an upper fusion surface and a lower fusion surface. The upper fusion surface and the lower fusion surface are configured for being in direct contact with vertebral endplates of a spine, and provided with anti-slip teeth. A center of the intervertebral fusion cage defines a bone graft window penetrating through the upper fusion surface and the lower fusion surface. The intervertebral fusion cage has a structure and mechanical properties similar to those of human cancellous bone and trabecular bone, and can increase the bone contact area and bone ingrowth space.

### CROSS-REFERENCE TO RELATED APPLICATION

This application claims priority to Chinese Patent Application No. 202510144041.6, filed Feb. 8, 2025, which is herein incorporated by reference in its entirety.

### TECHNICAL FIELD

The disclosure relates to the technical field of medical devices, and more particularly to an intervertebral fusion cage with a biomimetic trabecular bone structure and a fully porous structure manufactured by additive manufacturing.

### BACKGROUND

With the continuous development of medical technology, intervertebral fusion surgery is playing an increasingly important role in the treatment of spinal diseases. An intervertebral fusion cage, as a key implant in the intervertebral fusion surgery, directly affects the surgical outcome and patient recovery. Traditional intervertebral fusion cages are dense solid products made from materials such as titanium alloy and polyetheretherketone (PEEK) through mechanical processing. The traditional intervertebral fusion cages themselves do not have bone fusion capabilities and can only provide mechanical support, relying on the filling of autogenous bone or artificial bone in a bone graft window to achieve intervertebral fusion. Due to high elastic modulus of the dense structure of the materials, which does not match human bones, stress shielding occurs, leading to a higher incidence of cage subsidence and dislocation. A friction coefficient of the dense structure is relatively low, resulting in poor initial implant stability, and easily causing cage dislocation. Moreover, bone tissue cannot grow into the dense structure, which prolongs the vertebral fusion time and leads to poor clinical treatment effects.

In recent years, preparing porous intervertebral fusion cages by using additive manufacturing technologies such as selective laser melting (SLM) and electron beam melting (EBM) has attracted widespread attention from academia and industry. A porous structure can provide space for bone ingrowth, thereby increasing a bone contact area and a bone fusion volume, and low elastic modulus of the porous structure, which is closer to that of the human bones, reduces stress shielding and improves a success rate and long-term stability of the implant.

The porous intervertebral fusion cages currently still have some drawbacks in terms of structural design and fusion effects. For example, due to suboptimal porous structure design or poor strength of the material, in order to ensure mechanical properties, the porous intervertebral fusion cages can only be made as partially porous structures with dense solid frame works, resulting in a smaller bone contact area, less space for bone ingrowth, and poorer bone fusion performance. The unreasonable distribution of the porous structure may also lead to local stress concentration, affecting the mechanical reliability of the porous intervertebral fusion cages.

### SUMMARY

In view of the foregoing, the disclosure provides an intervertebral fusion cage with a biomimetic trabecular bone structure and a fully porous structure manufactured by additive manufacturing. Compared to dense solid frameworks in the related art, the intervertebral fusion cage of the disclosure has a structure and mechanical properties similar to those of cancellous bone and trabecular bone of the human body, which can increase a bone contact area and bone ingrowth space, has excellent initial and long-term stability, and can effectively reduce stress shielding, thereby enhancing bone fusion capability.

In order to achieve above purposes, the intervertebral fusion cage provided by the disclosure is applied in spinal fusion surgery, and has the biomimetic trabecular bone structure and the fully porous structure made of medical metal and manufactured using one of additive manufacturing technologies including SLM and EBM.

The intervertebral fusion cage includes a front end surface, a rear end surface, a first side surface, a second side surface, an upper fusion surface, and a lower fusion surface. The upper fusion surface and the lower fusion surface are configured for being in direct contact with vertebral endplates of a spine, and are provided with anti-slip teeth thereon, and a center of the intervertebral fusion cage defines a bone graft window penetrating through the upper fusion surface and the lower fusion surface.

In an embodiment, the intervertebral fusion cage is a cervical fusion cage applied in a cervical spine of the spine, and an angle between the upper fusion surface and the lower fusion surface of the cervical fusion cage is in a range of 0°-7°.

In an embodiment, the front end surface of the cervical fusion cage is a smooth plane structure to reduce friction and interference during an implantation process of the cervical fusion cage onto the vertebral endplates.

In an embodiment, the rear end surface of the cervical fusion cage is an arc-shaped structure configured to reduce compression and irritation of rear tissues and make the rear end surface fit closely with an instrument for implanting the cervical fusion cage onto the vertebral endplates to thereby increase an area of a striking surface of the cervical fusion cage during the implantation process of the cervical fusion cage, ensure that stress is evenly distributed on the cervical fusion cage when the cervical fusion cage is struck or subjected to a force, and prevent structural damage or deformation of the cervical fusion cage during the implantation process. The rear end surface of the cervical fusion cage defines an instrument hole extending through to the bone graft window, and the instrument hole is configured for insertion and fixation of the instrument.

In an embodiment, the first side surface and the second side surface of the cervical fusion cage are upright planes to enhance an overall strength of the cervical fusion cage and prevent deformation of the cervical fusion cage under side forces, and the first side surface and the second side surface are at a certain angle to reduce resistance during the implantation process of the cervical fusion cage.

In an embodiment, each of the first side surface and the second side surface defines holes or openings to reduce a weight of the cervical fusion cage and provide channels for bone growth and fusion inside and outside the cervical fusion cage.

In an embodiment, the intervertebral fusion cage is a lumbar fusion cage applied in a lumbar spine of the spine, and the front end surface of the lumbar fusion cage is a dual-bullet-shaped structure to reduce friction and interference with intervertebral space and surrounding tissues.

In an embodiment, the upper fusion surface and the lower fusion surface of the lumbar fusion cage are curved surface structures designed to increase a contact area between the lumbar fusion cage and the vertebral endplates and to make stress be evenly distributed across the upper fusion surface and the lower fusion surface when the lumbar fusion cage is subjected to a force.

In an embodiment, the rear end surface of the lumbar fusion cage is a plane structure, and each of the first side surface and the second side surface of the lumbar fusion cage defines a groove symmetrical to each other near the rear end surface.

In an embodiment, the first side surface and the second side surface of the lumbar fusion cage define side holes respectively, and the side holes are configured to provide channels for bone growth and fusion inside and outside the lumbar fusion cage.

From the technical solution described above, compared with the related art, the intervertebral fusion cage with the biomimetic trabecular bone structure and the fully porous structure manufactured by the additive manufacturing provided by the disclosure offers several advantages. The fully porous structure is precisely formed through the additive manufacturing technology, ensuring uniform distribution and connectivity of holes and channels. The biomimetic trabecular bone structure, which is three-dimensionally interconnected and microporous, rapidly absorbs blood through capillary action, promotes bone fusion and vascularization, and allows for effective exchange of nutrients and metabolic waste between the implant and the surrounding tissues, thereby optimizing the speed and degree of bone ingrowth and enhancing the formation and maturation of new bone. The intervertebral fusion cage has an elastic modulus and other mechanical properties that closely match those of the cancellous bone and trabecular bone of the human body, providing excellent biomechanical compatibility, eliminating the risk of stress shielding and bone resorption. With the outstanding load-bearing capacity, the intervertebral fusion cage can achieve immediate weight-bearing. The high friction coefficient between the intervertebral fusion cage and human bone enhances implant stability. Additionally, the intervertebral fusion cage has high porosity, significantly promoting bone and vascular ingrowth, further improving the fusion rate. Moreover, the intervertebral fusion cage has high toughness, high ductility and excellent fatigue resistance, and provides good radiopacity, facilitating postoperative assessment. The intervertebral fusion cage has high long-term stability, strong resistance to subsidence, and a highly anatomically matched shape, providing a higher fusion rate and implant stability. The comprehensive design of the structure of the intervertebral fusion cage provides superior biocompatibility and functional performance, meeting clinical demands for rapid fusion and long-term stability.

### BRIEF DESCRIPTION OF DRAWINGS

In order to provide a clearer explanation of technical solutions in embodiments of the disclosure or the related art, a brief introduction is provided below to the accompanying drawings required for the description of the embodiments or the related art. It is apparent that the accompanying drawings described below are only embodiments of the disclosure. For those skilled in the art, other drawings can be obtained based on the drawings described below without creative labor.

FIG. 1 illustrates a schematic three-dimensional diagram of a cervical fusion cage according to an embodiment 1 of the disclosure.

FIG. 2 illustrates a top view of the cervical fusion cage illustrated in FIG. 1.

FIG. 3 illustrates a sectional view along a line A-A of the cervical fusion cage illustrated in FIG. 2.

FIG. 4 illustrates a left-side view of the cervical fusion cage illustrated in FIG. 1.

FIG. 5 illustrates a front view of the cervical fusion cage illustrated in FIG. 1.

FIG. 6 illustrates a schematic three-dimensional diagram of a lumbar fusion cage according to an embodiment 2 of the disclosure.

FIG. 7 illustrates a front view of the lumbar fusion cage illustrated in FIG. 6.

FIG. 8 illustrates a top view of the lumbar fusion cage illustrated in FIG. 6.

FIG. 9 illustrates a partial close-up view of a porous structure of the cervical fusion cage illustrated in FIG. 1 and the lumbar fusion cage illustrated in FIG. 6.

FIG. 10A and FIG. 10B illustrate three-dimensional modeling diagrams of an intervertebral fusion cage of the disclosure; where FIG. 10A illustrates the three-dimensional modeling diagram of the cervical fusion cage, and FIG. 10B illustrates the three-dimensional modeling diagram of the lumbar fusion cage.

FIG. 11A and FIG. 11B illustrate actual product diagrams of the intervertebral fusion cage of the disclosure; where FIG. 11A illustrates the actual product diagram of the cervical fusion cage, and FIG. 11B illustrates the actual product diagram of the lumbar fusion cage.

FIG. 12 illustrates a load-displacement curve of the intervertebral fusion cage according to an embodiment of the disclosure.

FIG. 13 illustrates a dynamic compression F-N curve of the intervertebral fusion cage according to the embodiment of the disclosure.

FIG. 14 illustrates a bar chart showing percentage increase in bone contact area of intervertebral fusion cages of some embodiments of the disclosure compared to a traditional dense product.

FIG. 15 illustrates a bar chart showing available bone ingrowth pore volumes for the intervertebral fusion cages of some embodiments of the disclosure.

FIG. 16 illustrates an X-ray image from an animal experiment of the intervertebral fusion cage according to the embodiment of the disclosure.

FIG. 17 illustrates a bar chart showing measurement data of disc space heights (DSHs) at different implantation time points in the animal experiment of the intervertebral fusion cage according to the embodiment of the disclosure.

FIG. 18 illustrates a diagram showing histological staining images at different implantation stages in the animal experiment of the intervertebral fusion cage according to the embodiment of the disclosure.

FIG. 19 illustrates a bar chart showing bone fusion rates at different implantation stages in the animal experiment of the intervertebral fusion cage according to the embodiment of the disclosure.

Description of reference signs: 10: cervical fusion cage; 12: front end surface; 14: rear end surface; 16: upper fusion surface; 18: lower fusion surface; 20: second side surface; 22: first side surface; 24: anti-slip teeth; 26: bone graft window; 28: instrument hole; 44: groove; 48: side hole; 50: lumbar fusion cage.

### DETAILED DESCRIPTION OF EMBODIMENTS

The technical solutions in embodiments of the disclosure are clearly and completely described below in conjunction with the accompanying drawings. Apparently, the described embodiments are only a part of the embodiments of the disclosure, not all of them. The following description of an exemplary embodiment is merely illustrative and should not be construed as any limitation on the disclosure or its application or use. Based on the embodiments of the disclosure, all other embodiments obtained by those skilled in the art without creative labor are within the scope of protection of the disclosure.

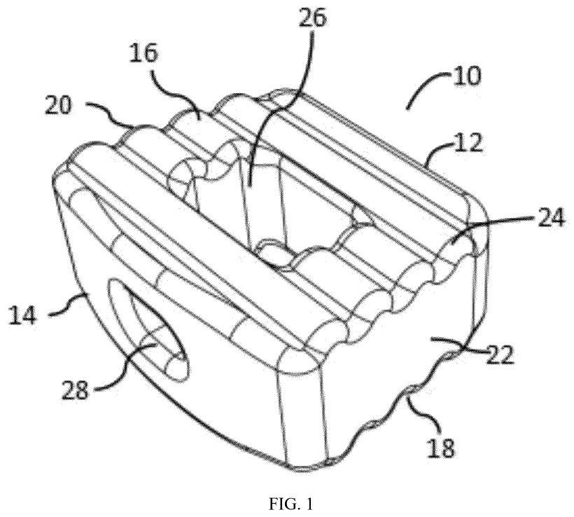

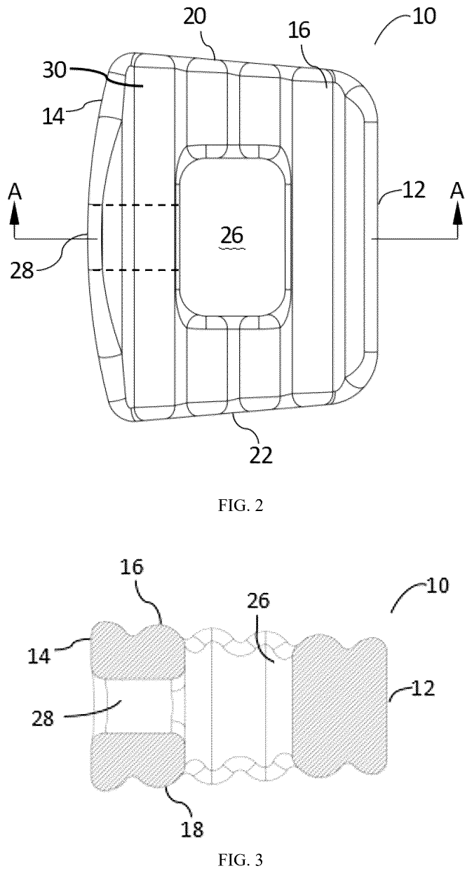

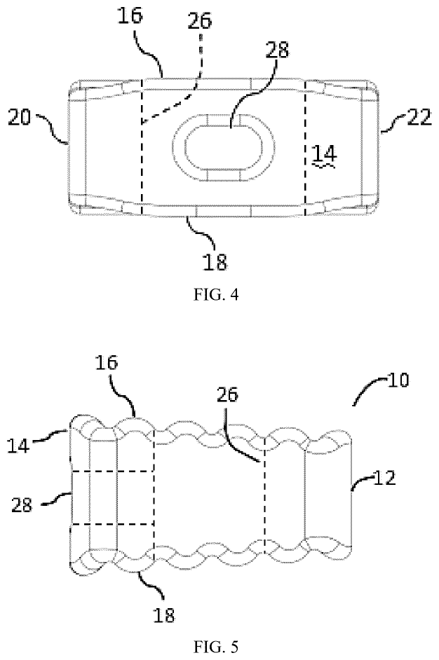

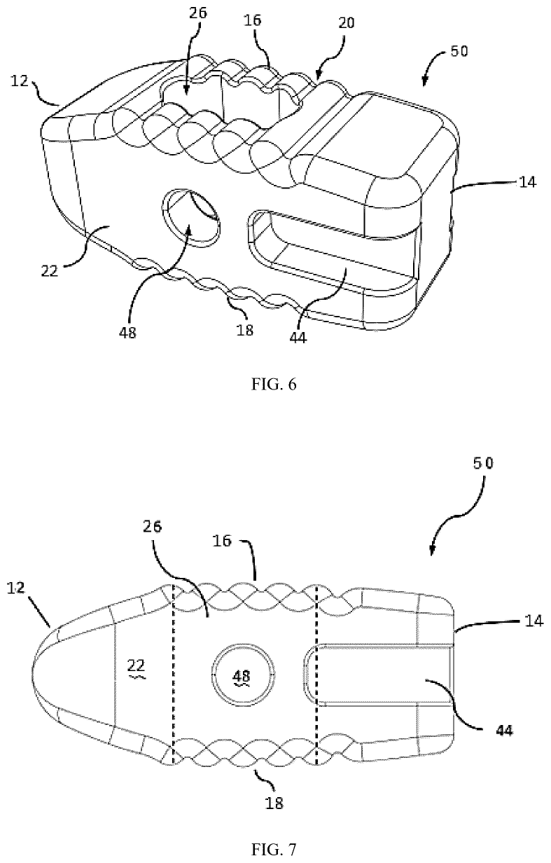

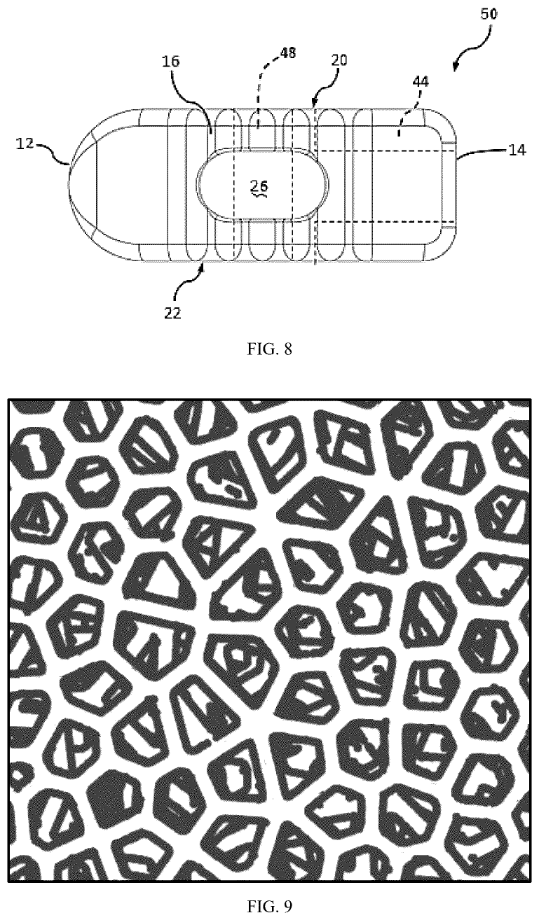

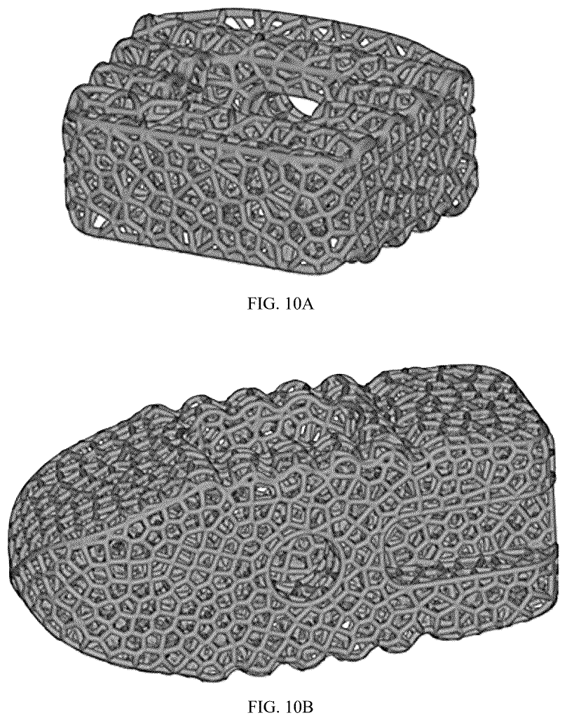

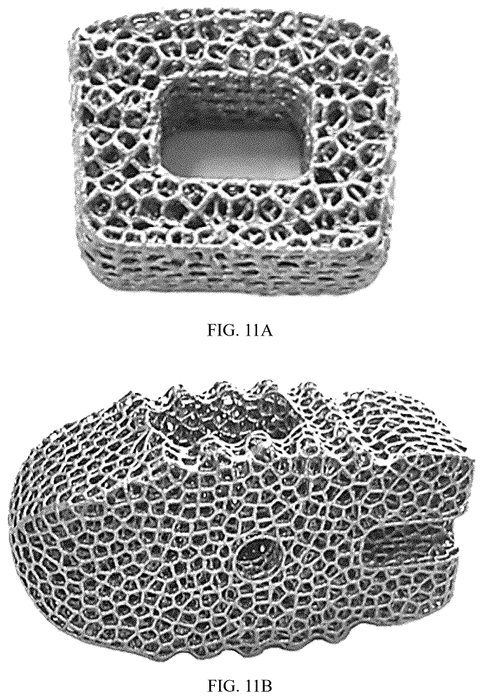

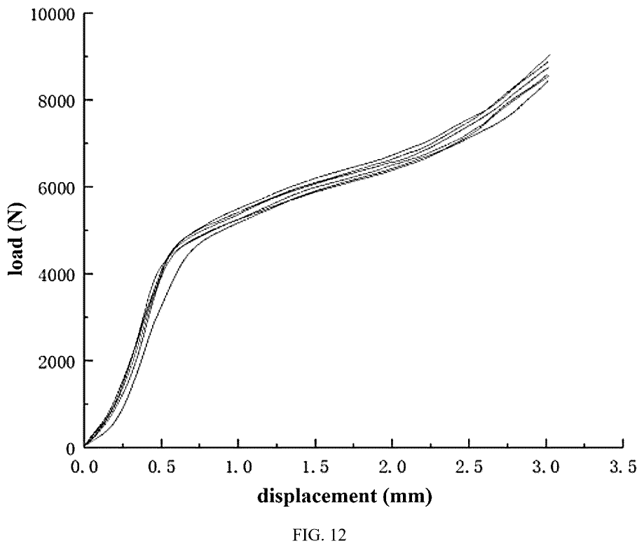

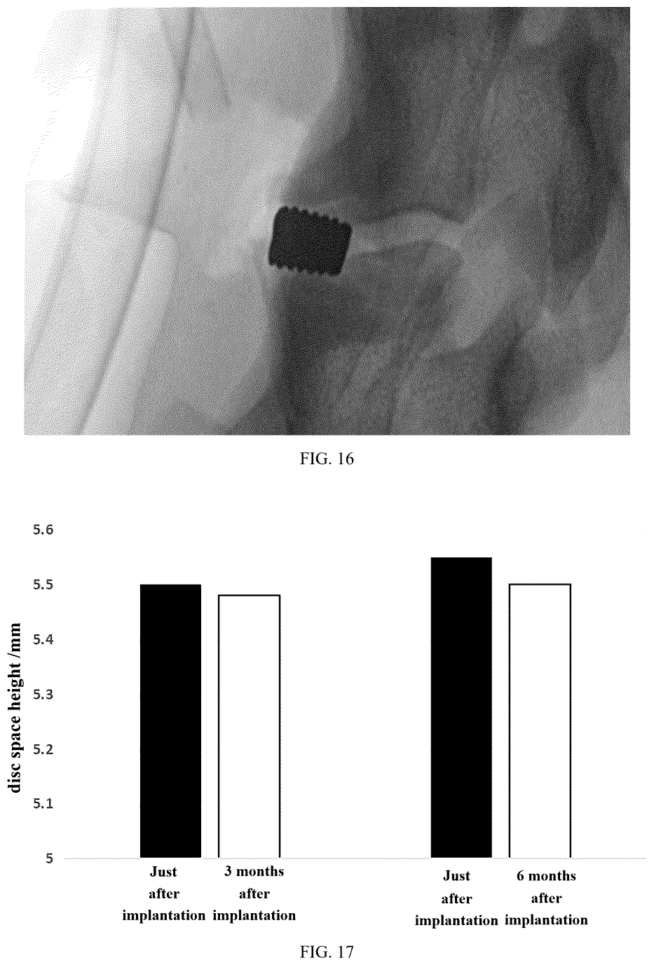

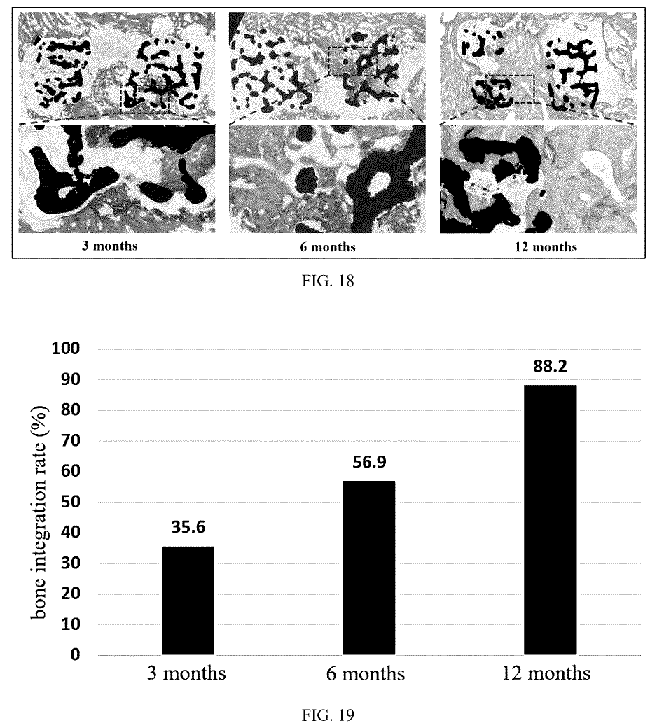

Referring to FIG. 1 to FIG. 11B, an intervertebral fusion cage with a biomimetic trabecular bone structure and a fully porous structure manufactured by additive manufacturing is illustrated. Please refer to FIG. 12 to FIG. 15, a load-displacement curve, a dynamic compression F-N curve, i.e., a stress level (F)-number of cycles to failure (N) curve, a bar chart showing percentage increase in bone contact area of the intervertebral fusion cage of the disclosure compared to a traditional dense product, and a bar chart showing the available bone ingrowth pore volume of the intervertebral fusion cage of the disclosure are shown. Referring to FIG. 16 to FIG. 19, related drawings of an animal experiment of the intervertebral fusion cage of the disclosure are shown.

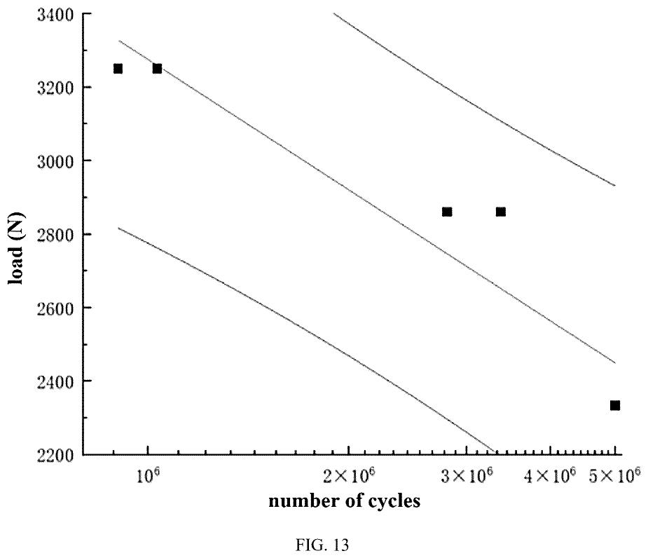

The intervertebral fusion cage with the biomimetic trabecular bone structure and the fully porous structure manufactured by the additive manufacturing provided by the disclosure is applied in fusion surgery of a spine. The spine includes structures such as a cervical spine and a lumbar spine. The intervertebral fusion cage has the biomimetic trabecular bone structure and the fully porous structure made of medical metal and manufactured using one of additive manufacturing technologies including SLM and EBM. The fully porous structure is formed in one step through the additive manufacturing technologies, ensuring uniform distribution and connectivity of the pores, thereby optimizing the speed and degree of bone ingrowth. As shown in FIG. 12 to FIG. 13, the biomimetic fully porous structure has high ductility and mechanical reliability, with excellent fatigue resistance. The biomimetic fully porous structure of the intervertebral fusion cage has a static compressive stiffness greater than 10000 newtons per millimeter (N/mm), a yield strength greater than 4000 newtons (N), and under 5 million cycles of loading, a compressive fatigue limit strength greater than 2000 N, effectively reducing the risk of fracture failure of the implant during long-term use.

The intervertebral fusion cage includes: a front end surface 12, a rear end surface 14, a first side surface 22, a second side surface 20, an upper fusion surface 16, and a lower fusion surface 18. The upper fusion surface 16 and the lower fusion surface 18 are configured for being in direct contact with vertebral endplates of the spine, and are provided with anti-slip teeth 24 thereon. The anti-slip teeth 24 can effectively increase the friction between the implant and the vertebral endplates after implantation, preventing the implant from shifting or rotating postoperatively, thereby enhancing the stability of the intervertebral fusion cage and increasing the success rate of fusion. A center of the intervertebral fusion cage defines a bone graft window 26 penetrating through the upper fusion surface 16 and the lower fusion surface 18. The bone graft window 26 is configured to be filled with autograft or artificial bone material. The opening of the bone graft window 26 is precisely designed to be sufficiently large to accommodate the required bone graft material while maintaining the structural integrity of the implant. The through-window design of the bone graft window 26 maximizes the contact area between the bone graft material and the endplates, promotes bone ingrowth and fusion, accelerates the postoperative healing process, and ensures the integration of new bone with the implant, thereby improving the fusion outcome. The exact position of the bone graft window 26 can be optimized based on specific conditions to ensure a maximum contact area between the bone material and upper and lower endplates, thereby increasing the success rate of bone fusion.

Specifically, the medical metal includes, but is not limited to, tantalum, titanium, tantalum alloys, and titanium alloys. the medical metal possesses excellent mechanical properties, corrosion resistance and biocompatibility, which are key to successful long-term implantation. The porous metal structure is designed to mimic natural bone trabeculae, promoting bone integration and enhancing biological fixation. Each of the anti-slip teeth 24 is arc-shaped with a depth of 0.5 millimeters. The anti-slip teeth 24 can reduce tissue damage caused during the implantation process of the intervertebral fusion cage, improve stress distribution, and reduce the risk of wear and debris generation during long-term use of the implant, thereby extending the service life of the implant.

### Embodiment 1

As shown in FIG. 1 to FIG. 5, the intervertebral fusion cage is a cervical fusion cage 10 applied in a cervical spine of the spine. An overall structure of the cervical fusion cage 10 fully considers anatomical features and biomechanical requirements of the cervical spine, aiming to provide excellent stability and fusion outcomes.

The front end surface 12 of the cervical fusion cage 10 is a plane structure, configured to provide a base surface for implantation and positioning. The front end surface 12 of the cervical fusion cage 10 is designed to be smooth without protrusions to reduce friction and interference during the implantation process, facilitate the smooth entry of the cervical fusion cage 10 into the intervertebral space, and minimize damage to the intervertebral disc tissue.

The upper fusion surface 16 and the lower fusion surface 18 are key areas to be in direct contact with the vertebral endplates. The design of the upper fusion surface 16 and the lower fusion surface 18 directly affects the stability of the implant and the bone fusion outcome. To accommodate the physiological structure of the vertebral endplates in different patients, an angle between the upper fusion surface 16 and the lower fusion surface 18 is arranged in a range of 0°to 7°. The angle is designed based on the normal physiological curvature of the cervical spine, ensuring better conformity and stability, thereby promoting the restoration of the natural physiological curvature of the spine.

The rear end surface 14 is an arc-shaped structure, specifically designed to improve the conformity of the implant and the convenience of surgical manipulation. The rear end surface 14 with the arc-shaped structure conforms to the anatomical curve of the cervical spine, to reduce compression and irritation on rear tissues and enhance the conformity and stability of the implant during surgery. More importantly, the arc-shaped design allows the rear end surface 14 to closely fit with the instrument for implanting the cervical fusion cage 10 onto the vertebral endplates, to thereby increase an area of a striking surface of the cervical fusion cage 10 during the implantation process. This conformity provides greater operational stability for surgeons, and ensures that stress is evenly distributed on the cervical fusion cage 10 when the cervical fusion cage 10 is struck or subjected to a force, thereby preventing structural damage or deformation of the cervical fusion cage 10 during the implantation process. The rear end surface 14 defines an instrument hole 28 extending through to the bone graft window 26, making it convenient to insert and fix the instrument during the surgery. The design of the instrument hole 28 is optimized to ensure precise insertion of the instrument without affecting the filling of the bone graft material or the progression of bone fusion.

The first side surface 22 and the second side surface 20 are planes at a certain angle, providing overall structural support and lateral stability to the implant. The first side surface 22 and the second side surface 20 are designed as upright planes to enhance an overall strength of the cervical fusion cage 10 and prevent deformation of the cervical fusion cage 10 under side forces. The angle between the first side surface 22 and the second side surface 20 is designed to reduce resistance during the implantation process of the cervical fusion cage 10. Additionally, the first side surface 22 and the second side surface 20 can be designed with extra holes or openings as needed, to reduce a weight of the implant and provide additional pathways for bone ingrowth.

### Embodiment 2

As shown in FIG. 6 to FIG. 8, the intervertebral fusion cage is a lumbar fusion cage 50 applied in a lumbar spine of the spine. An overall structure of the lumbar fusion cage 50 fully considers anatomical features and biomechanical requirements of the lumbar spine, aiming to provide excellent stability and fusion outcomes.

The front end surface 12 of the lumbar fusion cage 50 uses a design of a dual-bullet-shaped structure, which is a streamlined structure can effectively reduce implantation resistance, making the positioning of the lumbar fusion cage 50 easier during surgery and thereby reducing the complexity and duration of the surgery operation. The dual-bullet shape not only simplifies the implantation process, but also reduces the risk of tissue damage by reducing friction and interference with intervertebral space and surrounding tissues, thereby improving the conformity of the lumbar fusion cage 50.

The upper fusion surface 16 and the lower fusion surface 18 are designed as curved surfaces, and a radius of the curved surface is 150 millimeters. The design allows for precise matching with the lumbar vertebral endplates, enhancing initial implant stability and reducing the risk of postoperative implant displacement and subsidence. The curved surface structure can increase the contact area between the implant and the endplates, improving stress distribution by making the stress on the lumbar fusion cage 50 distributed more uniformly, thereby reducing the risk of endplate collapse under pressure.

The rear end surface 14 is a plane structure, and each of the first side surface 22 and the second side surface 20 of the lumbar fusion cage 50 defines a groove 44 symmetrical to each other near the rear end surface 14. The groove 44 is configured to allow for gripping the instrument. The design of the groove 44 provides secure grip points, allowing surgeons to more precisely manipulate and position the implant during surgery, ensuring stability and accuracy in the implantation process. The presence of the grooves 44 symmetrical to each other also enhances the ease of operation, reducing potential errors during surgery.

The first side surface 22 and the second side surface 20 define side holes 48 respectively. The design of the side holes 48 facilitates bone growth and fusion inside and outside the lumbar fusion cage 50. The side holes 48 not only reduce the weight of the implant, but also provide additional channels for bone tissue growth, promoting integration between the implant and surrounding bone tissue. The side holes 48 increase the air permeability of the implant, aiding in the vascularization process and further accelerating healing.

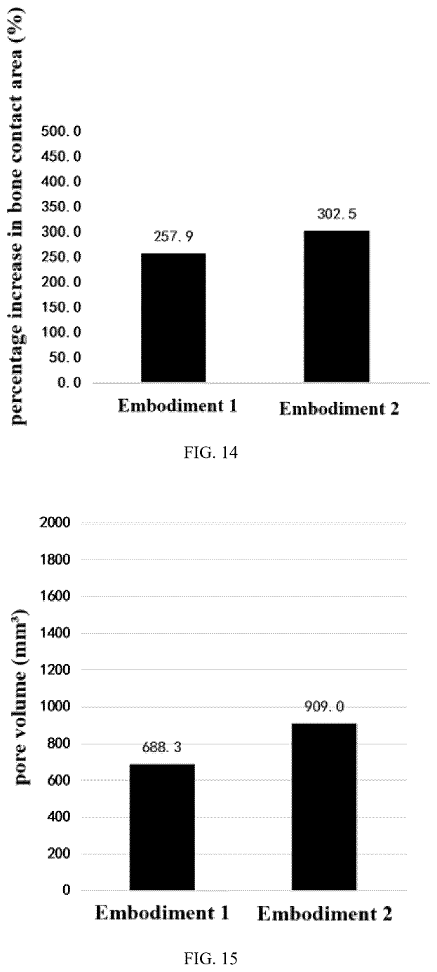

As shown in FIG. 9, the intervertebral fusion cage of the disclosure uses a fully porous design. Compared with traditional fusion cages that contain dense solid parts, the intervertebral fusion cage of the disclosure significantly increases a volume available for bone ingrowth and substantially enhances the bone fusion outcome. As shown in FIG. 14 and FIG. 15, it is calculated that, compared with traditional dense fusion cage products, the fully porous structure of the disclosure increases the surface area by approximately 1.5 to 3.5 times and provides a pore volume of about 400 to 1500 cubic millimeters (mm³) available for bone ingrowth. This design offers a larger area for bone integration and more space for bone cells to attach and grow, thereby accelerating the bone fusion process. The pore structure units of the intervertebral fusion cage of the disclosure are irregular polygons, randomly arranged in all directions, exhibiting mechanical isotropy. This design better adapts to the complex biomechanical environment of the spine, providing uniform stress distribution and good biomechanical stability. The porous structure of the intervertebral fusion cage has an average pore size in a range of 240-800 micrometers, an average pore strut diameter of 200-500 micrometers, and a porosity of 50% to 90%, consistent with the structural characteristics of human cancellous bone. This porous structure of the intervertebral fusion cage of the disclosure not only helps to reduce the overall elastic modulus of the implant, bringing it closer to the mechanical properties of natural bone, but also provides ample space and channels for bone cell ingrowth and vascular formation, further promoting the integration and fusion of bone tissue with the implant.

To verify the bone fusion performance of the intervertebral fusion cage with the biomimetic trabecular bone and fully porous structure manufactured by the additive manufacturing of the disclosure, animal experiments are conducted. The bone fusion outcomes are assessed at different time points (3 months, 6 months, and 12 months) using medical imaging technology, DSH measurement data, and histological staining results. Radiological examinations, including X-ray and computed tomography (CT) scans, are performed at 3, 6 and 12 months postoperatively, respectively. Quantitative analysis of trabecular bone formation and changes in bone density is used to preliminarily determine the formation of new bone. As shown in FIG. 16 to FIG. 19, imaging results demonstrate that the intervertebral fusion cage of the disclosure exhibits good bone fusion outcomes at 3, 6, and 12 months postoperatively. The trabecular bone structure gradually increased, and the bone integration interface is clear, indicating effective bone integration between the new bone and the support material. The DSH test results show no significant differences in DSH just after implantation, and at 3 months and 6 months postoperatively, indicating that the implant did not undergo significant displacement and exhibits excellent stability. Hematoxylin-eosin (HE) staining is used, and histological analysis is performed on the bone fusion area at 3, 6, and 12 months postoperatively. The results show visible implant material in the bone defect area, osteoblasts at the edge of the bone defect, and new bone formation at the boundary of the bone defect, and new bone formation is also observed between the implant material and the pores. At 6 months postoperatively, the bone fusion reached grade D, i.e., the bone tissue bridged the entire fusion area and achieved a density being (or greater than) at least the density obtained intraoperatively. At 12 months postoperatively, the bone fusion rate (i.e., the bone integration rate) exceeded 85%.

The above description of the illustrated embodiments enables those skilled in the art to implement or use the disclosure. Various modifications to the embodiments are apparent to those skilled in the art, and the general principles defined herein can be implemented in other embodiments without departing from the spirit or scope of the disclosure. Therefore, the disclosure will not be limited to the embodiments shown herein, but will be within the widest scope consistent with the principles and novel features disclosed herein.

1. An intervertebral fusion cage with a biomimetic trabecular bone structure and a fully porous structure manufactured by additive manufacturing, applied in spinal fusion surgery, wherein the intervertebral fusion cage has the biomimetic trabecular bone structure and the fully porous structure made of medical metal and manufactured using one of additive manufacturing technologies comprising selective laser melting and electron beam melting, and the intervertebral fusion cage comprises:
a front end surface (12), a rear end surface (14), a first side surface (22), a second side surface (20), an upper fusion surface (16), and a lower fusion surface (18);
wherein the upper fusion surface (16) and the lower fusion surface (18) are configured for being in direct contact with vertebral endplates of a spine, and are provided with anti-slip teeth (24) thereon, and a center of the intervertebral fusion cage defines a bone graft window (26) penetrating through the upper fusion surface (16) and the lower fusion surface (18); and
wherein each of the anti-slip teeth (24) on the upper fusion surface (16) extends from the first side surface (22) to the second side surface (20), adjacent two of the anti-slip teeth (24) on the upper fusion surface (16) are connected, and the anti-slip teeth (24) on the upper fusion surface (16) fully cover the upper fusion surface (16).

2. The intervertebral fusion cage as claimed in claim 1, wherein the intervertebral fusion cage is a cervical fusion cage (10) applied in a cervical spine of the spine, and an angle between the upper fusion surface (16) and the lower fusion surface (18) of the cervical fusion cage (10) is in a range of 0°-7°.

3. The intervertebral fusion cage as claimed in claim 2, wherein the front end surface (12) of the cervical fusion cage (10) is a smooth plane structure to reduce friction and interference during an implantation process of the cervical fusion cage (10) onto the vertebral endplates.

4. The intervertebral fusion cage as claimed in claim 2, wherein the rear end surface (14) of the cervical fusion cage (10) is an arc-shaped structure configured to reduce compression and irritation of rear tissues and make the rear end surface (14) fit closely with an instrument for implanting the cervical fusion cage (10) onto the vertebral endplates to thereby increase an area of a striking surface of the cervical fusion cage (10) during an implantation process of the cervical fusion cage (10), ensure that stress is evenly distributed on the cervical fusion cage (10) when the cervical fusion cage (10) is struck or subjected to a force, and prevent structural damage or deformation of the cervical fusion cage (10) during the implantation process; and the rear end surface (14) of the cervical fusion cage (10) defines an instrument hole (28) extending through to the bone graft window

(26) and the instrument hole (28) is configured for insertion and fixation of the instrument.

5. The intervertebral fusion cage as claimed in claim 2, wherein the first side surface (22) and the second side surface (20) of the cervical fusion cage (10) are upright planes to enhance an overall strength of the cervical fusion cage (10) and prevent deformation of the cervical fusion cage (10) under side forces, and the first side surface (22) and the second side surface (20) are at an angle to reduce resistance during an implantation process of the cervical fusion cage (10).

6. The intervertebral fusion cage as claimed in claim 1, wherein the intervertebral fusion cage is a lumbar fusion cage (50) applied in a lumbar spine of the spine, and the front end surface (12) of the lumbar fusion cage (50) is a dual-bullet-shaped structure configured to reduce friction and interference with intervertebral space and surrounding tissues.

7. The intervertebral fusion cage as claimed in claim 6, wherein the upper fusion surface (16) and the lower fusion surface (18) of the lumbar fusion cage (50) are curved surface structures designed to increase a contact area between the lumbar fusion cage (50) and the vertebral endplates and to make stress be evenly distributed across the upper fusion surface (16) and the lower fusion surface (18) when the lumbar fusion cage (50) is subjected to a force.

8. The intervertebral fusion cage as claimed in claim 6, wherein the rear end surface (14) of the lumbar fusion cage (50) is a plane structure, and each of the first side surface (22) and the second side surface (20) of the lumbar fusion cage (50) defines a groove (44) symmetrical to each other near the rear end surface (14) of the lumbar fusion cage (50).

9. The intervertebral fusion cage as claimed in claim 6, wherein the first side surface (22) and the second side surface (20) of the lumbar fusion cage (50) define side holes (48) respectively, and the side holes (48) are configured to provide channels for bone growth and fusion inside and outside the lumbar fusion cage (50).

10. The intervertebral fusion cage as claimed in claim 6, wherein each of the upper fusion surface (16) and the lower fusion surface (18) of the lumbar fusion cage (50) is a curved surface, and a radius of the curved surface is 150 millimeters.

11. The intervertebral fusion cage as claimed in claim 1, wherein a static compressive stiffness of the intervertebral fusion cage is greater than 10000 newtons per millimeter (N/mm), a yield strength of the intervertebral fusion cage is greater than 4000 newtons (N), and under 5 million cycles of loading, a compressive fatigue limit strength of the intervertebral fusion cage is greater than 2000 N.

12. The intervertebral fusion cage as claimed in claim 1, wherein the medical metal comprises: tantalum, titanium, tantalum alloys, or titanium alloys.

13. The intervertebral fusion cage as claimed in claim 1, wherein each of the anti-slip teeth (24) is arc-shaped with a depth of 0.5 millimeters.

14. The intervertebral fusion cage as claimed in claim 1, wherein the fully porous structure has an average pore strut diameter in a range of 200 micrometers to 500 micrometers, and a porosity in a range of 50% to 90%.

15. An intervertebral fusion cage with a biomimetic trabecular bone structure and a fully porous structure manufactured by additive manufacturing, comprising:
a front end surface (12), a rear end surface (14), a first side surface (22), a second side surface (20), an upper fusion surface (16), and a lower fusion surface (18);
wherein the first side surface (22) is connected to the front end surface (12), the rear end surface (14), the upper fusion surface (16) and the lower fusion surface (18); the second side surface (20) is connected to the front end surface (12), the rear end surface (14), the upper fusion surface (16) and the lower fusion surface (18); the upper fusion surface (16) is connected to the front end surface (12) and the rear end surface (14); and the lower fusion surface (18) is connected to the front end surface (12) and the rear end surface (14);
wherein the upper fusion surface (16) and the lower fusion surface (18) are provided with anti-slip teeth (24) thereon, and a center of the intervertebral fusion cage defines a bone graft window (26) penetrating through the upper fusion surface (16) and the lower fusion surface (18);
wherein the rear end surface (14) is an arc-shaped structure, the first side surface (22) and the second side surface (20) are upright planes, and an angle between the upper fusion surface (16) and the lower fusion surface (18) is in a range of 0°-7°; and
wherein each of the anti-slip teeth (24) on the upper fusion surface (16) extends from the first side surface (22) to the second side surface (20), adjacent two of the anti-slip teeth (24) on the upper fusion surface (16) are connected along sides thereof that extend from the first side surface (22) to the second side surface (20), and the anti-slip teeth (24) on the upper fusion surface (16) fully cover the upper fusion surface (16); and the fully porous structure provides a pore volume of 400 to 1500 cubic millimeters (mm³) available for bone ingrowth.

16. The intervertebral fusion cage as claimed in claim 15, wherein the front end surface (12) is a smooth plane structure.

17. The intervertebral fusion cage as claimed in claim 15, wherein the rear end surface (14) defines an instrument hole (28) extending through to the bone graft window (26), and the instrument hole (28) is configured for insertion and fixation of an instrument for implementing the intervertebral fusion cage.

18-19. (canceled)

20. The intervertebral fusion cage as claimed in claim 1, wherein the fully porous structure provides a pore volume of 400 to 1500 mm³ available for bone ingrowth
